(19) United States
(12) Patent Application Publication
(10) Pub. No.: US 2023/0105909 A1
(43) Pub. Date: Apr. 6, 2023

(54) Method and System For Generating An ECG Signal

(71) Applicant: Fraunhofer Gesellschaft zur Förderung der angewandten Forschung e.V., München (DE)

(72) Inventor: Marian Häscher, Rostock (DE)

(21) Appl. No.: 17/962,469

(22) Filed: Oct. 8, 2022

Related U.S. Application Data
(63) Continuation of application No. PCT/EP2021/059165, filed on Apr. 8, 2021.

(30) Foreign Application Priority Data
Apr. 9, 2020 (DE) 102020204650.6
Jun. 24, 2020 (DE) 102020207845.9

(51) Int. Cl. A61B 5/11; A61B 5/318; A61B 5/00

(52) U.S. Cl. CPC A61B 5/1102; A61B 5/318; A61B 5/7253; A61B 5/7264; A61B 5/742; A61B 5/7221

(57) ABSTRACT

A method for generating electrocardiogram (ECG) signals includes detecting at least one cardiac motion induced signal. The at least one cardiac motion induced signal is a seismocardiography (SCG) signal. The method includes transforming the at least one detected cardiac motion induced signal into at least one ECG signal. Multiple channel-specific signals of a multi-channel ECG signal are determined by the transformation from the at least one SCG signal.

### CROSS-REFERENCE TO RELATED APPLICATIONS

This application is a continuation of PCT App. No. PCT/EP2021/059165 filed 8 Apr. 2021, which claims priority to German App. No. 102020204650.6 filed 9 Apr. 2020 and German App. No. 102020207845.9 filed 24 Apr. 2020.

### FIELD

The present application relates to a method and a system for determining an ECG signal.

### BACKGROUND

ECG signals are required in a variety of application, especially in diagnostic applications. It is not possible to record a normal surface ECG contactlessly. Electrodes are needed here to record the voltage potentials on the surface of the chest. Long-term recording with these electrodes may cause skin irritation and rashes. There is also a risk, especially with such measurements, that an electrode may become detached and thus undesirably impair the quality of the ECG signal. Some patients (e.g. premature babies or burns victims) also have very sensitive and thin skin, which entails an increased risk of infections and skin injuries during contact measurements or even prohibits the use of electrodes altogether, thus preventing the important measurements of vital data.

In addition, the ECG devices used to date do not allow unbroken and continuous measurement over a period of 24 hours. Thus, intermittent cardiac pathologies might be overlooked and thus not diagnosed and not treated.

So far, there is no medically valid alternative: The ECG is either applied anyway and skin damage and risk of infection are accepted after consideration, or the measurement is omitted, which means that vital data is unable to be monitored or diagnostics are not possible.

In the case of continuous recording which extends beyond a period of 24 hours, expensive event recorders are used in individual cases after consideration of the cost-benefit ratio, the costs of the recorders usually not being covered by health insurance. Event recorders record signals with increased accuracy in the event of an anomaly and are otherwise in an energy-saving mode.

Single-channel ECG solutions also exist in the field of portable computers (wearables). These offer simple recording functionality and fast signal analysis, especially to detect serious heart diseases. However, they are also cost-intensive and—if they support the ECG function—must be purchased additionally. The ECG function in particular cannot simply be retrofitted in old devices via a software update. The medical and especially diagnostic validity of the previous single-channel ECG solutions is not yet available.

Also known is the detection of seismocardiography signals (SCG signal), which may also be referred to as a precordial motion signal. The precordium may refer to a part of the chest wall in front of the heart. Thus, the precordial motion signal may include information about the motion of this part of the chest wall. In particular, such a signal includes information about movements, especially oscillations, of the precordium caused by heart movements. From such signals, even movements of heart valves, e.g. the aortic valve or the mitral valve, may be detected and corresponding characteristics identified. While the electrical stimuli visualized during the ECG examination represent the electrical stimuli that occur prior to any muscle movement within the cardiac cycle, the SCG signal represents the resulting movements measured at the precordial position. This approach uses, for example, widely available inertial sensors such as accelerometers or gyroscopes. However, pressure or radar sensors may also be used.

Also known is the detection of phonocardiography signals, these being audio signals generated by the reception of sound waves, the sound waves being caused by cardiac motion. Also known is the detection of ballistocardiography signals, these being the vibration of the whole body caused by the cardiac motion. Ballistocardiograms may be recorded all over the body and are therefore not fixed to a particular measurement point. Much of the research in the field of mobile and portable seismocardiography focuses on extracting vital parameters such as heart rate, heart rate variability or respiratory rate. Although these parameters provide valuable information about the user's health status, doctors are able to extract much more information from the rhythmology and morphology of the electrocardiogram (ECG).

Machine learning techniques are also known, including in cardiology. a plurality of well-known methods use convolutional autoencoders to compress health data by reducing the complexity or noise in biological signals, as has been shown for EEG and ECG signals.

Especially for the detection of diseases in ECG signals, neural networks have proven to be a powerful tool, e.g. for the detection of atrial fibrillation, for the automated detection of myocardial infarctions, as well as for the detection of arrhythmias.

In addition to the application of neural networks to the analysis of ECG data, there are a number of publications that apply machine learning to signals from other types of sensors. CNNs (convolutional neural networks) may be used to estimate heart rates from PPG sensors (photoplethysmography sensors) or to automatically detect cardiovascular disease from SCG data.

WO 2014/036436 A1, which discloses an apparatus and a method for monitoring the heart of a patient, is known from the prior art. This publication discloses a mobile telephone with an acceleration sensor. It is further disclosed that a corresponding apparatus may also comprise a plurality of electrodes integrated into or arranged on the mobile phone. The apparatus may be placed on the patient's chest to detect electrical signals and chest vibrations due to a heartbeat. Measurements may produce an SCG (seismocardiogram) and an ECG (electrocardiogram).

Furthermore, a large number of solutions are known in the prior art that allow the temporally parallel acquisition of an SCG and an ECG, for example from EP 2 704 634 A1, EP 3 135 194 A1, US 2018/360338 A1 and WO 2018/231444 A2.

WO 2019/138327 A1 also discloses a portable ECG device, which may additionally comprise an SCG sensor. EP 3 461 401 A1 also discloses an apparatus for detecting ECG signals and SCG signals.

Classification methods for classifying a state of a vehicle occupant as a function of biometric data are also known. For example, DE 10 2019 201 695 A1 discloses a neural network used in a vehicle component to determine the stress level or arousal level of a vehicle occupant. US 2019/0088373 A1 discloses the use of artificial intelligence and machine learning in healthcare.

### SUMMARY

The technical problem therefore arises of creating a method and a system for creating an ECG signal which allows the ECG signal to be generated accurately and as reliably as possible, avoiding in particular a contacting arrangement of electrodes having the disadvantages explained above and enabling reliable long-term recording of an ECG signal.

The solution of the technical problem results from the subjects with the features of the independent claims. Further advantageous embodiments of the invention result from the subjects with the features of the dependent claims.

A method is proposed for generating an ECG (electrocardiography) signal, wherein at least one cardiac-motion-induced signal is detected. A cardiac-motion-induced signal may refer to a signal caused by cardiac motion. It is also possible that a plurality of cardiac-motion-induced signals are detected, in particular also signals of different types. This will be explained below. A cardiac-motion-induced signal may in particular be an SCG signal (seismocardiography signal) or a PCG signal (phonocardiography signal) or a BCG signal (ballistocardiography signal). This cardiac-motion-induced signal may be generated by a suitable detection means. For example, the SCG signal may be generated by a suitable SCG detection means, the PCG signal may be generated by a suitable PCG detection means, and the BCG signal may be generated by a suitable BCG detection means.

For example, such an SCG detection means may comprise at least one acceleration sensor, e.g. a MEMS acceleration sensor, in particular a MEMS gyroscope, or a radar sensor, in particular a Doppler radar sensor. As previously explained, the SCG signal contains or encodes information about cardiac motion. Such acceleration sensors may be uniaxial or triaxial piezoelectric acceleration sensors or MEMS acceleration sensors, triaxial MEMS acceleration sensors or gyroscopes, laser Doppler vibrometers, microwave Doppler radar sensors, or a so-called Airborne ultrasound surface motion camera (AUSMC). A PCG detection means may in particular comprise a microphone, in particular a microphone of a mobile terminal device such as a mobile phone or a laser microphone. A BCG detection means may, for example, comprise at least one pressure sensor, e.g. a pressure sensor formed as a load cell.

According to the invention, the at least one detected cardiac-motion-induced signal is transformed into at least one ECG signal. Example transformation processes are explained in more detail below. As explained in more detail below, it is also conceivable to transform the at least one cardiac-motion-induced signal into a plurality of ECG signals. It is also possible—as explained in more detail below—that a plurality of detected cardiac-motion-induced signals are transformed into one or more ECG signals.

Thus, it has surprisingly been found that a cardiac-motion-induced signal and an ECG signal have a comparable information content with regard to heart activity, since ECG signals also contain or encode information about cardiac motion. Conversely, a cardiac-motion-induced signal also contains information about electrical activities of the heart. Since cardiac-motion-induced signals are regularly incomprehensible to users without appropriate processing, because they are usually not used in everyday clinical practice, especially for diagnosis, and their interpretation is usually not part of medical training, the transformation may produce an ECG signal that is generally meaningful to a larger group of people, which increases the medical applicability, e.g. for diagnostic purposes. Furthermore, ECG signals require the previously explained mechanical contacting of the patient's skin to enable reliable generation. This is advantageously not absolutely necessary for the acquisition of cardiac-motion-induced signals.

Preferably, the cardiac-motion-induced signal is thus detected without contact, i.e. without mechanical contact of a patient by a corresponding sensor. For example, this may be done by placing the detection means at a distance from the patient, for example in a mattress on which the patient lies or in a seat in which the patient sits. If the detection means includes, for example, a radar sensor, it is only necessary to arrange the detection means in such a way that the patient or a chest area of the patient is arranged in the detection area of the radar sensor.

However, it is also possible that the cardiac-motion-induced signal is detected by a sensor that mechanically contacts or is arranged in or on the patient for detection. Thus, it is possible that the detection means is integrated in a pacemaker, in particular in a rate-adaptive pacemaker. A pacemaker may comprise such a detection means, in particular a detection means in the form of an acceleration sensor, in order to adjust a rate of a patient's heartbeat as a function of the signal detected by the detection means, e.g. in order to adapt it to the current state of movement as well as pulse demand. To make this possible, activities are detected as a function of output signals from the acceleration sensors and, for example, the rate of the heartbeat increases accordingly as the load increases (e.g. when changing from walking to climbing stairs). The acceleration sensors used for this purpose may also be used to detect a cardiac-motion-induced signal.

A signal detected by such a detection means may then be transmitted to a computing means, e.g. wirelessly via suitable methods for data transmission, the computing means then carrying out the transformation. This (external) computing means may be, for example, a computing means of a mobile terminal device. Alternatively, it is conceivable that the pacemaker comprises a computing means, which then carries out the transformation. Such a computing means of the pacemaker may be integrated in the pacemaker in the form of an embedded system. For example, the computing means may be formed as an integrated circuit which is specifically designed to carry out the transformation. This integrated circuit may, for example, provide the functionality of a neural network.

The use of a detection means integrated in a pacemaker advantageously allows the use of sensors already present and located close to the heart, which results in a good signal quality of the cardiac-motion-induced signals. This in turn improves the measurement accuracy and thus also the accuracy of the ECG signal generated in accordance with the invention. Furthermore, due to the extended use of an already certified pacemaker, a simple certification of a system for generating an ECG signal as a medical device which comprises the detection means of the pacemaker is also made possible.

The transformation thus transforms the at least one cardiac-motion-induced signal, which represents for example precordial movements, sound waves caused by these movements or whole-body movements and transforms them into a signal that represents or replicates electrical potentials. The transformation into an ECG signal may be a direct transformation. The transformation may also comprise a plurality of partial transformations, wherein, for example, the cardiac-motion-induced signal is transformed into an intermediate signal by a first partial transformation and in a further partial transformation the intermediate signal is transformed into the ECG signal. It is of course possible that more than two partial transformations are also carried out. The proposed method advantageously results in a simple and reliable generation of an ECG signal, which may be carried out in particular, but not necessarily, without contact, i.e. without mechanical contact of the skin by a detection means. Furthermore, the proposed method enables reliable long-term recording of ECG signals, in particular over a period of more than 24 hours, since cardiac-motion-induced signals may be recorded and then transformed without any problems over such a period of time, in particular because the problem of unintentional detachment of electrodes is avoided.

Furthermore, it is possible to advantageously implement and thus retrofit the claimed method on existing devices that have a detection means suitable for sensing cardiac-motion-induced signals, thereby enabling these devices to generate an ECG signal. For example, mobile phones typically include acceleration sensors. These may be used to generate SCG signals, for example by placing a mobile phone on a patient's chest and acquiring output signals from the acceleration sensor. These output signals may then be transformed into an ECG signal by the proposed transformation. Also, a microphone of a mobile phone may be used to generate PCG signals.

In a further embodiment, the at least one cardiac-motion-induced signal is an SCG signal. This advantageously results in a reliable provision of an ECG signal, as SCG signals are able to be reliably generated. Alternatively, the cardiac-motion-induced signal is a PCG signal. Since this comprises a broad frequency spectrum, this advantageously results in an accurate generation of an ECG signal.

Alternatively, the cardiac-motion-induced signal is an ECG signal. Since this may be measured on the whole body, it is advantageous to have a flexible detection and thus generation of an ECG signal.

It is conceivable that a plurality of, in particular different, cardiac-motion-induced signals are detected, e.g. a plurality of SCG signals, a plurality of BCG signals or a plurality of PCG signals. It is also conceivable that at least two different signals of the signal set comprising SCG signal, PCG signal and BCG signal are detected, wherein the at least one ECG signal is then generated by a transformation of these different signals into the at least one ECG signal. It is also conceivable that a fused cardiac-motion-induced signal is generated from the different cardiac-motion-induced signals and this is then transformed into at least one ECG signal.

In a further embodiment, the at least one cardiac-motion-induced signal is transformed into at least one channel-specific signal of a multi-channel ECG. It is also conceivable that exactly one channel-specific signal or selected, but not all, channel-specific signals or all channel-specific signals of a multi-channel ECG is/are determined by the transformation of the at least one cardiac-motion-induced signal. For example, one or more channel-specific signal(s) of an ECG may be determined from one or more SCG signal(s), one or more PCG signal(s), one or more BGK signal(s) or at least two different signals of the signal set comprising SCG, PCG and BCG signal(s). The channel-specific signal of a multi-channel ECG determined by transformation represents/simulates the ECG signal derived in/at a predetermined body region by a correspondingly arranged electrode. The multi-channel ECG may be, for example, a 6-channel or a 12-channel ECG. This advantageously increases the usability of the ECG signals generated in this way, since in particular the generation of a multi-channel ECG allows a simpler or better analysis and thus diagnosis.

In another embodiment, the transformation is carried out using a model generated by machine learning. The term “machine learning” includes or refers to methods for determining the model based on training data. Thus, it is possible to determine the model by methods for supervised learning, for which purpose the training data, i.e. a training data set, comprise input data and output data. In this case, cardiac-motion-induced signals may be provided as input data, and the ECG signals corresponding to these cardiac-motion-induced signals are provided as output data.

In particular, input and output data of such training data may be generated by simultaneously generating cardiac-motion-induced signals and ECG signals, wherein these simultaneously generated data then form the input and output data for the training. Methods and apparatuses for the simultaneous generation of such data are known from the prior art, which was explained in the introduction to the description. For example, the model is able to learn the relationship between the seismocardiogram, ballistocardiogram or phonocardiogram and the electrocardiogram. Such methods for supervised learning are known to a person skilled in the art. It is also conceivable that unsupervised learning methods are used to determine the model.

After the model has been created, i.e. after the training phase, the model parameterized in this way may be used in the so-called inference phase to then generate the ECG signals to be determined from input data in the form of cardiac-motion-induced signals, i.e. to carry out the proposed transformation. This results in a reliable and high-quality generation of ECG signals. It is possible that the model is determined in a user- or patient-unspecific and/or detection-means-unspecific manner, wherein the model thus determined is then used to carry out the transformation for a specific user and/or a specific detection means. This may mean that the model is not determined individually for a specific user and/or for a specific detection means, but may then be used in the inference phase for an individual user and/or an individual detection means.

It is therefore possible that the model does not have to be trained anew for each user and/or each detection means. In particular, it may be trained once, preferably with a suitably large data set (training phase) and then used as a model independently of the user and/or detection means, e.g. for all users (inference phase). Thus, an applicability of the method is advantageously improved, since in particular a specific training does not have to take place for each user and/or each detection means. For example, the same model may be used to transform signals generated by different detection means. Here, the suitable data set preferably comprises data generated for at least a predetermined number of different sick or healthy persons and/or for at least a predetermined number of physiologies and/or for at least a predetermined number of different diseases. However, it may of course be necessary to train the model with input data of the same characteristic, i.e. only with SCG signals, PCG signals or BCG signals, but using different detection means or different configurations of a detection means to acquire these signals of the same characteristic. Of course, it is also possible that the model is determined in a user-specific and/or detection-means-specific manner.

Suitable mathematical algorithms for machine learning include: Decision Tree-based methods, Ensemble methods (e.g. Boosting, Random Forrest)-based methods, Regression-based methods, Bayesian Methods (e.g. Bayesian Belief Networks)-based methods, Kernel methods (e.g. Support Vector Machines)-based methods, Instance- (e.g. k-Nearest Neighbor)-based methods, Association Rule Learning-based methods, Boltzmann Machine-based methods, Artificial Neural Networks (e.g. Perceptron)-based methods, Deep Learning (e.g. Convolutional Neural Networks, Stacked Autoencoders)-based methods, Dimensionality Reduction-based methods, Regularization Methods-based methods.

To train e.g. a neural network, a large amount of training data is required regularly to ensure a desired quality of transformation. The amount of training data may depend on factors such as the complexity of the underlying problem, the required accuracy and the desired adaptability of the network to be trained. The application domain, i.e. the domain in which the network is to be used, is often the most important element in determining these factors and thus in determining the amount of training data. With appropriate prior knowledge about the domain, it is possible to prepare data for training the network that lead to a faster convergence to the optimal solution, or enable such a convergence in the first place and thus require less training data.

The proposed method is used in a medical environment. Thus, a high degree of accuracy is desirable. In addition, there is a comparatively high complexity, since ECG signals and cardiac-motion-induced signals differ from each other due to the different sensors used to detect them. However, this usually requires a large amount of data to train a neural network. A possible step to reduce the required amount of data is to filter the training data, in particular the input data and/or the output data. In particular, input and output data of a training data set may be generated by simultaneously generating cardiac-motion-induced signals and ECG signals and then filtering them prior to training. This reduces the memory requirements as well as the computing time and/or power needed to determine/generate the model. Thus, it is possible to filter the training data with a filter, in particular a band-pass filter, e.g. a Butterworth filter, in order to attenuate high-frequency as well as low-frequency components in the training data. For example, a first, lower cut-off frequency of a bandpass filter may be 0.5 Hz and a further, upper cut-off frequency may be 200 Hz. It is also conceivable to use high-pass and/or low-pass filters or other filters (e.g. polynomial filters) to filter out the corresponding unwanted frequencies from the training data. Alternatively, the generated signals may also be used unfiltered for training.

In a further embodiment, an error function for determining a deviation between an ECG signal determined by transformation and a reference ECG signal is evaluated for generating the model, wherein different signal sections of the ECG signal determined by transformation and/or of the reference ECG signal and/or of the deviation (of the deviation signal) are weighted differently during the evaluation of the error function. Thus, an ECG signal-specific error function may be used. The reference ECG signal may represent ground truth and may be, for example, an ECG signal acquired in parallel with the input data (i.e., a cardiac-motion-induced signal) and acquired with a known, e.g., electrode-based, ECG detection means. The error function is used to determine or quantify a deviation between the result of the transformation, i.e. the ECG signal determined by the transformation, and the ground truth. This deviation then influences the determination, in particular the training, of the model for the transformation by machine learning, in particular the determination of a neural network, wherein the model is adapted, for example, in such a way that the deviation is reduced. Here, the deviation may be determined, for example, as a mean square deviation or a mean absolute deviation.

It is possible that, in order to determine the deviation, different signal sections of the ECG signal determined by transformation or of the reference ECG signal are weighted differently and all signal sections of the remaining signal are weighted equally. Preferably, all signal sections of the ECG signal determined by transformation and all signal sections of the reference ECG signal are weighted equally to determine the deviation, but different sections of the signal representing the deviation are weighted differently. A weighted section in the deviation signal may be a section that corresponds in time to a predetermined (relevant) section in the ECG signal determined by transformation and/or in the reference ECG signal. The different weighting of different signal sections in at least one of the signals may advantageously improve a quality of the model and thus also the signal quality of the ECG signal determined by transformation. The different weighting of different signal sections allows in particular characteristic and thus relevant sections of the ECG signal to be weighted higher than less relevant ones. Relevant ECG signal sections may be identified by an expert, for example by selecting signal sections using an input means. Alternatively, however, it is also conceivable to carry out an automated detection of relevant signal sections, for example via suitable detection methods that identify e.g. sections with predetermined signal properties. In such detection methods, for example, a phasor transformation may be carried out. In this case, sections with predetermined signal properties may be assigned predetermined weights. A relevant section in a signal may be a P-wave signal section, a QRS complex signal section and/or a T-wave section.

In another embodiment, the transformation is carried out using a neural network. For example, the neural network may be formed as an autoencoder or as a convolutional neural network (CNN) or as an RNN (recurrent neural network) or as an LSTM network (long short-term memory network) or as a neural transformer network or as a combination of at least two of the networks mentioned. Such a neural network, in particular the neural network formed as an autoencoder, may be trained in this case by means of the previously explained training data, wherein the transformation of a detected cardiac-motion-induced signal into the ECG signal may then be carried out after the training.

The formation of the neural network as an autoencoder offers the advantage that the computational effort required for the transformation is low, which means that the transformation may be carried out reliably and quickly in a simple manner by embedded systems and portable terminal devices such as mobile phones.

The formation as CNN advantageously allows a reduction in the complexity of the network and is thus suitable for devices with low computing power. This applies to both the training phase and the inference phase. It is also advantageous that the time required for training is short for CNNs, in particular shorter than for LSTM networks, which also require comparatively higher computing power. However, the formation as an LSTM network is particularly well suited for the analysis of time series, since its architecture takes into account the reference to time dependencies. This results in a high quality of the transformation and the ECG signal determined therewith.

In an alternative embodiment, the transformation is carried out by means of a predetermined mathematical model or by means of a predetermined transformation function. This may be predetermined by a user, for example. In particular, it is possible to suitably parameterize mathematical models for the transformation of cardiac-motion-induced signals into ECG signals. This advantageously results in an alternative reliable and temporally fast generation of ECG signals.

In a further embodiment, the at least one cardiac-motion-induced signal is detected contactlessly. If a plurality of such signals are detected, exactly one, a plurality of but not all, or all signals may be detected contactlessly. This and corresponding advantages have been explained previously.

In a further embodiment, the at least one cardiac-motion-induced signal is filtered prior to the transformation and then the filtered cardiac-motion-induced signal is transformed into an ECG signal. In particular, the filtering may be a high-pass or band-pass or band-stop filtering. A corresponding filter for carrying out the filtering may in particular be a Butterworth or polynomial filter. If the filtering is a high-pass filtering, a cut-off frequency of the high-pass filter may be, for example, in a range of 5 Hz to 8 Hz to reliably reduce effects of motion artefacts on the cardiac-motion-induced signal. If the filtering is bandpass filtering, a first cut-off frequency may be, for example, in a range of from 5 Hz (inclusive or exclusive) to 8 Hz (inclusive or exclusive) and a further cut-off frequency may be in a range of from 30 Hz (inclusive or exclusive) to 35 Hz (inclusive or exclusive) to also reliably reduce the effect of motion artefacts that are, for example, outside the range of from 8 Hz to 30 Hz. The filtering may be carried out in particular by Butterworth filters or polynomial filters. This advantageously results in a more accurate determination of the ECG signal, especially when the patient is moving during the detection of the cardiac-motion-induced signal.

In a further embodiment, the at least one cardiac-motion-induced signal is generated by a detection means of a device. Example detection means have been explained previously. The device here refers to a unit comprising the detection means. For example, the device may be a mobile phone or a tablet PC. However, other embodiments of such a device are of course also conceivable. Further, the transformation is carried out by a computing means of the device. In other words, the device comprises both the detection means and the computing means. A computing means may in this case be in the form of a microcontroller or integrated circuit or may comprise such a microcontroller or integrated circuit. Thus, it is possible to carry out the transformation or a partial transformation by a programmable or hard-wired component, in particular a chip (e.g. ASIC, FPGA). Such a component may then carry out the transformation in stand-alone mode or as part of a system-in-package (SiP). It is also possible to integrate the means for carrying out the transformation, e.g. as an SoC (system-on-a-chip), directly into a sensor for detecting the cardiac-motion-induced signal (e.g. MEMS acceleration sensor) or into another electronic component. This advantageously results in a centralized acquisition and generation of ECG signals, for example on a terminal device, in particular a mobile terminal device.

In addition to the explained means for signal processing, the device may also comprise means for signal storage, means for signal transmission, and means for display. However, it may also be possible that the device does not comprise any or all of the means explained. In this case, the detected cardiac-motion-induced signal may be transmitted to a further device comprising one or more further means. The ECG signal generated in this way may therefore also be visualized, for example by a display means of the device. Also, the ECG signal may be stored, for example by a memory means of the device. It is further possible to transmit the ECG signal from the device to an external system, for example via a suitable communication means of the device.

Alternatively, the cardiac-motion-induced signal is transmitted from the detection means to a computing means external to the device, the transformation being carried out by this computing means external to the device. The computing means external to the device may in particular be a server means or the computing means of a further device. In this case, too, the cardiac-motion-induced signal may be visualized, for example by a display means of the device, for which purpose the ECG signals determined by the transformation carried out by the computing means external to the device are transmitted back to the device. Of course, it is also possible to visualize the ECG signal determined in this way by a display means external to the device. For this purpose, the ECG signal may be transmitted to the corresponding further device for display. Furthermore, the ECG signal determined in this way may be stored or processed further, for example by the memory means or computing means external to the device or a further memory or computing means (external to the device).

The computing means external to the device may be or may form a server means of a network, in particular of the internet. In particular, the computing means external to the device may be part of a server means that offers cloud-based services.

The transmission to the computing means external to the device may preferably take place wirelessly, for example by means of suitable transmission methods. However, it is of course also possible to design the transmission so as to be wired. The computing means of a device which also comprises the detection means is thus advantageously not overloaded by the transformation. In particular, it is thus possible to carry out the detection of the cardiac-motion-induced signal by devices that provide comparatively low computing power, whereby corresponding transformation and, if necessary, further processing may then be carried out by other computing means with comparatively higher computing power.

In a further embodiment, the at least one cardiac-motion-induced signal is generated by a detection means of a device and the ECG signal determined by transformation is displayed on a display means of the device or on an external display means, for example a display means of a further device. For example, it is possible for the cardiac-motion-induced signal to be transmitted from the device to a computing means external to the device and for the transformation to be carried out there, with the ECG signal determined in this way then being transmitted to a further device, for example a further mobile radio telephone, and then being displayed on its display means. The ECG signal may also be transmitted back to the device and displayed by its display means. Also, the ECG signals may be displayed by a display in a browser, especially if the computing means external to the device is a server means or part thereof.

This makes it possible to carry out remote monitoring based on the ECG signals generated in accordance with the invention.

In a further embodiment, a functional test of a detection means is carried out prior to transforming the at least one cardiac-motion-induced signal, wherein the cardiac-motion-induced signal is only transformed if a functional capability is detected. A functional capability may be detected, for example, if the detection means generates a temporally varying output signal. If a temporally constant output signal is generated or if the output signal does not deviate more than a predetermined amount from a constant output signal, a lack of functional capability may be detected. Alternatively or cumulatively, a functional capability may be detected if the output signal has characteristics that deviate more than a predetermined amount from predetermined noise characteristics—in particular, white noise characteristics. If this is the case, a functional capability may be detected. If this is not the case, a lack of functional capability may be detected. A lack of functional capability may also be detected if a sampling rate of the output signal deviates from a target sampling rate and/or a quantization of the output signal deviates from permissible quantification values. In the case of a lack of functional capability, no transformation is able to be carried out. The transformation is thus advantageously only carried out if it may be assumed that the detection means is functional. This reduces energy consumption when carrying out the method.

Alternatively or cumulatively, prior to transforming the at least one cardiac-motion-induced signal, a signal quality of the detected signal is determined, wherein the cardiac-motion-induced signal is transformed only if the signal quality is greater than or equal to a predetermined measure. For example, a signal quality may be a signal-to-noise ratio or a quantity representing that ratio. If this ratio is greater than a predetermined measure, the transformation may be carried out. Also, a signal quality may be greater than or equal to a predetermined measure if a deviation between a predetermined reference waveform and a detected waveform in a section of the cardiac-motion-induced signal is less than or equal to a predetermined measure. This may also be referred to as a so-called template comparison. Here, a classical waveform of a cardiac-motion-induced signal, i.e. the reference waveform, may be determined and stored. Then, using methods known to a person skilled in the art, a deviation between the signal waveform of the detected cardiac-motion-induced signal and the reference signal waveform may be determined.

A signal quality may also be determined using suitable models such as neural networks. Training data for such models may be generated by assigning a quality measure representing the signal quality to a cardiac-motion-induced signal, e.g. by a user or in a (partially) automated manner. This assignment may also be referred to as annotation. Here, the cardiac-motion-induced signal forms the input data and the quality measure the output data of the training data set. Such training data may be generated in particular by generating and annotating cardiac-motion-induced signals in different spatial positions of the detection means, in particular relative to the heart, with different SNR, under different ambient conditions, in different patient motion states, etc.

It is further possible that such a model, in particular a neural network, for determining the signal quality is also used for filtering the training data for determining the model generated by machine learning for the transformation. In this case, only those cardiac-motion-induced signals for which the signal quality is higher than a predetermined measure are used as input data for training the model for transformation. By evaluating the signal quality as a condition for carrying out the transformation, it may be advantageously ensured that a reliable and high-quality transformation takes place.

It is also possible that, in addition to the signal quality, a quality-reducing cause is determined via suitable models such as neural networks. Training data for such models may be generated by assigning the quality-reducing cause to a cardiac-motion-induced signal, e.g. by a user or in a (partially) automated manner. This assignment may also be referred to as annotation. In this case, the cardiac-motion-induced signal forms the input data and the cause the output data of the training data set. Quality-reducing causes may be, for example, the presence of artefacts, the arrangement of the detection means in spatial positions that are unfavorable for the detection, in particular relative to the heart, and/or the presence of unfavorable environmental or movement conditions. If a quality-reducing cause is able to be determined in this way, the user may be informed of the cause, for example via a display means. In addition, the user may be given a recommendation for action to remedy the cause.

Further alternatively or cumulatively, prior to transforming the at least one cardiac-motion-induced signal, a position, i.e. a spatial position and/or orientation, of the detection means relative to the heart is determined, wherein the cardiac-motion-induced signal is transformed only if the position corresponds to a predetermined position or deviates by less than a predetermined amount therefrom. For example, it is possible for the cardiac-motion-induced signal to have predetermined signal properties only when the position corresponds to a predetermined position or deviates by less than a predetermined measure therefrom. Thus, signal characteristics of the cardiac-motion-induced signal may be determined and compared to the predetermined signal characteristics. If the deviation is less than a predetermined measure, the position corresponds to the predetermined position or deviates by less than a predetermined amount therefrom.

It is also possible that the position is determined via suitable models such as neural networks. Training data for such models may be generated by assigning the position to a cardiac-motion-induced signal, e.g. by a user or in a (partially) automated manner. This assignment may also be referred to as annotation. In this case, the cardiac-motion-induced signal forms the input data and the position the output data of the training data set. Such training data may be generated in particular by generating cardiac-motion-induced signals in different spatial positions of the detection means, in particular relative to the heart, and by annotating them accordingly. If a position is able to be determined, the user may be informed of the position, in particular its correctness, for example via a display means. In addition, the user may be given a recommendation for action to change the position if it deviates from the predetermined position by more than the predetermined measure.

By determining the position as a condition for carrying out the transformation, it may be advantageously ensured that the transformation is reliable and of a high quality. For example, a situation may be avoided in which a detection means for detecting the cardiac-motion-induced signal is not arranged in the correct way, for example an acceleration sensor does not rest on a body surface, and thus a quality of the ECG signal determined by transformation is reduced.

It is conceivable that for determining the functional capability and/or the signal quality and/or the position, the cardiac-motion-induced signals intended for transformation are evaluated, wherein these are used for transformation if a functional capability is detected and/or the signal quality is greater than or equal to the predetermined measure and/or the position does not deviate from the predetermined position by more than a predetermined measure. Alternatively, the functional capability and/or the signal quality and/or the position may be determined on the basis of cardiac-motion-induced signals not intended for transformation, wherein a further detection of the cardiac-motion-induced signal is carried out for transformation if a functional capability is detected and/or the signal quality is greater than or equal to the predetermined measure and/or the position does not deviate from the predetermined position by more than a predetermined measure.

The ECG signal may in particular be the ECG signal of a human, i.e. a signal for/in human medical applications. However, the method may also be used to generate an ECG signal of an animal, i.e. to generate a signal for/in veterinary applications. Thus, an ECG acquisition in animals, in particular without electrodes, advantageously allows a great reduction of stress in animals in which an ECG is to be acquired, e.g. for diagnostic purposes.

For example, 24-hour Holter ECGs may be recorded in animals, especially horses or dogs, but this is a major stress factor for the animals due to the necessary visit to the veterinarian and the subsequent wiring of the animal. This may be particularly problematic when the pathologies are episodic, since in dogs, for example, stress provides for undetectable clinical signs. However, the method according to the invention does not require a person skilled in the art to generate an ECG signal, in particular to apply the electrodes, and it allows contactless measurement, in particular also of multi-channel ECG signals.

For example, a detection means may be integrated into a harness or chest strap that is put on the animal. Thus, such a detection means may be purchased and put on by the pet owner. For example, an acceleration sensor in the harness/chest strap is able to detect the animal's cardiac-motion-induced signals and to enable the explained transformation. Likewise, the method may be used in order to be applied by veterinarians in routine examinations. Since animals usually show symptoms of a disease of the cardiovascular system at a very late stage, this method may allow diagnoses already at an earlier stage of such diseases. The veterinarian is able to record a resting ECG of the animal by placing a suitable detection means or device with a detection means, e.g. a smartphone, without having to apply a large number of electrodes. This also eliminates the disadvantages of using electrodes on the animal (e.g. artefacts on the ECG signal due to fur interfering with the electrodes). This concept of examination may also be applied to domestic fish, e.g. koi, as well as horses and camels, which is particularly interesting in the field of competitive sports for such animals.

In the livestock sector, medical monitoring is regularly carried out on a reduced basis according to cost or effort, e.g. by a veterinarian diagnosing by cohort. However, ECG monitoring would also provide the doctor with valuable information regarding animal welfare (e.g. fitness, health status, stress assessment, early detection of bacterial infections such as streptococci). Until now, however, ECG monitoring of individual animals using current methods has been very time-consuming and expensive. The proposed method offers an inexpensive and simple way of monitoring, e.g. if the cardiac-motion-induced signal is recorded contactlessly, e.g. by using radar sensors. Animals are thus able to be monitored contactlessly and thus also hygienically. This monitoring would be conceivable for farm animals such as pigs and ruminants, but also fish. The proposed method may also be used in animal research. It may also be used in zoos and animal parks to ensure the health of the animals with as little stress as possible.

A system for generating an ECG signal is further proposed, the system comprising at least one detection means for detecting at least one cardiac-motion-induced signal and at least one computing means. As previously explained, the detection means and the computing means may each be part of a device. However, it is also conceivable that the detection means and the at least one computing means are each parts of different devices. It is also conceivable that the system comprises a plurality of detection means for detecting a plurality of cardiac-motion-induced signals.

In accordance with the invention, the at least one detected cardiac-motion-induced signal may be transformed into at least one ECG signal by means of the computing means. For this purpose, it may be necessary to transmit the signal detected by the detection means to the computing means, for example by means of transmission means.

Advantageously, the system allows a method for generating an ECG signal according to one of the embodiments described in this disclosure to be carried out with the corresponding advantages mentioned. Thus, the system is configured such that such a method may be carried out with the system.

In a further embodiment, the detection means is integrated into an incubator. For example, the detection means in this case may comprise a Doppler radar sensor and may be arranged on a ceiling of the incubator, in particular in such a way that a chest area of the patient lying on a mattress of the incubator is arranged in the detection range of the radar sensor. Alternatively, the detection means may comprise or may be formed as an acceleration sensor arranged in/on the floor of the incubator or in/on the mattress of the incubator.

Alternatively, the detection means may be arranged in a bed, in particular a hospital bed. If the detection means is formed as a Doppler radar sensor, for example, it may be arranged under the mattress or above the bed, for example attached to a bed frame. Also conceivable is the previously explained formation of the detection means as an acceleration sensor, which is arranged in/on the mattress or in/on the floor of the bed. It is also possible to form the detection means as a pressure sensor which is arranged in/on the mattress of the bed. Further alternatively, the detection means is integrated into a vehicle seat. In this case, a detection means formed as a Doppler radar sensor may, for example, be arranged in/on a seat back. A detection means in the form of a pressure sensor may be arranged in/on the seat back. The same applies to a detection means formed as an acceleration sensor. Further alternatively, the detection means is integrated in a pacemaker. Further alternatively, the detection means is integrated in an animal accessory, e.g. a chest strap, a halter, a collar or the like.

Thus, a system for generating an ECG signal is also described, comprising an incubator, wherein the detection means is arranged in/on the incubator or in/on a mattress of the incubator. A system comprising a bed is further described, wherein the detection means is arranged in/on the bed or in/on a mattress of the bed. Further, a system for generating an ECG signal is described which additionally comprises a vehicle seat, wherein the detection means is arranged in/on the vehicle seat. Further, a system for generating an ECG signal is described which additionally comprises a cardiac pacemaker, wherein the detection means is arranged in/on the cardiac pacemaker. Further, a system for generating an ECG signal is described which additionally comprises an animal accessory, wherein the detection means is arranged in/on the animal accessory. Of course, other applications are also conceivable. Also described is an incubator, a bed, a mattress, a vehicle seat, a pacemaker and an animal accessory comprising at least the detection means of such a system.

### BRIEF DESCRIPTION OF THE DRAWINGS

The invention is explained in more detail with the aid of example embodiments. The figures show:

FIG. 1 is a schematic representation of a method according to the invention for determining an ECG signal,

FIG. 2 is a schematic block diagram of a system according to the invention for generating an ECG signal according to a first embodiment,

FIG. 3 is a schematic representation of a system according to the invention for generating an ECG signal according to a further embodiment,

FIG. 4 is a schematic flow diagram of a method according to the invention,

FIG. 5 is a schematic representation of a system for generating an ECG signal according to a further embodiment,

FIG. 6 is a schematic representation of a system for generating an ECG signal according to a further embodiment,

FIG. 7 is a schematic representation of a system for generating an ECG signal according to a further embodiment,

FIG. 8 is a schematic representation of an example application of the method according to the invention,

FIG. 9 is a schematic representation of a system for generating an ECG signal with an incubator,

FIG. 10 is a schematic representation of a system for generating an ECG signal with a hospital bed,

FIG. 11 is a schematic representation of a system for generating an ECG signal with a vehicle seat,

FIG. 12 is a schematic representation of a method according to the invention in a further embodiment,

FIG. 13 is a schematic representation of the generation/training of the neural network shown in FIG. 12,

FIG. 14 is a schematic representation of synchronized ECG and SCG signals,

FIG. 15 is a schematic representation of an ECG signal determined by transformation and an ECG signal recorded by electrodes,

FIG. 16a is a schematic representation of a dog harness with a detection means of a system for generating an ECG signal,

FIG. 16b is a schematic representation of a horse holster with a detection means of a system for generating an ECG signal,

FIG. 17 is a schematic representation of a pacemaker with a system for generating an ECG signal, and

FIG. 18 is an example representation of weightings of different signal sections.

### DETAILED DESCRIPTION

In the following, like reference signs denote elements with like or similar technical features.

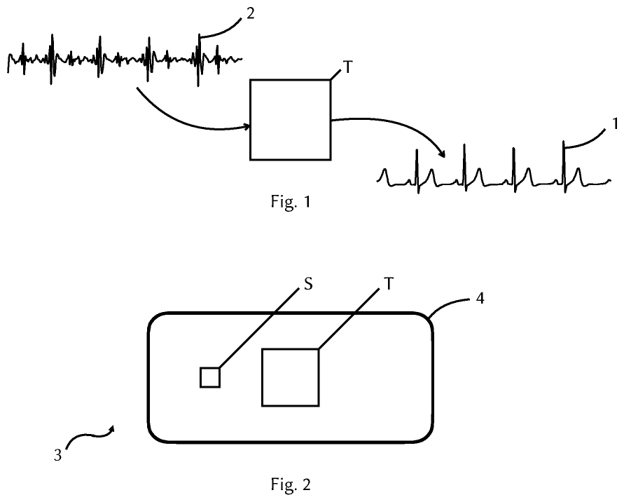

FIG. 1 shows a schematic representation of a method for generating an ECG signal 1. Here, a cardiac-motion-induced signal in the form of an SCG signal 2 is detected. This may be done by means of an SCG detection means S, which will be explained in more detail below. Then, a transformation means T, which may in particular be formed as a computing means or may comprise a computing means, transforms the detected SCG signal 2 into an ECG signal 1. Alternatively or cumulatively, a PCG signal, e.g. by a PCG detection means, may also be detected as a cardiac-motion-induced signal and transformed into an ECG signal 1. Further alternatively or cumulatively, a BCG signal, e.g. by a BCG detection means, may also be detected as a cardiac-motion-induced signal and transformed into an ECG signal 1.

FIG. 2 shows a schematic block diagram of a system 3 for generating an ECG signal 1 (see FIG. 1). The system 3 comprises an SCG detection means S and at least one transformation means T, which is formed as a computing means. It is shown that the ECG detection means and the transformation means are part of a device 4, for example a mobile phone.

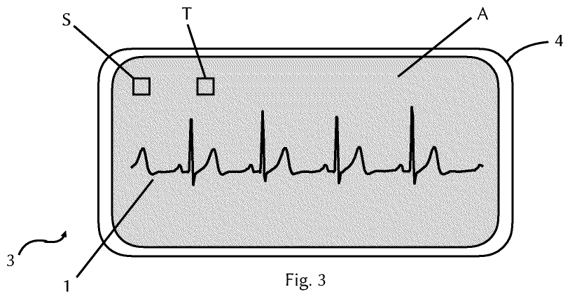

FIG. 3 shows a representation of the system 3 for generating an ECG signal 1 in accordance with a further embodiment. As explained above, the system 3 comprises an SCG detection means S and a transformation means T in the form of a computing means. A display means A is also shown, on which the ECG signal 1 is visualized. It is shown here that the SCG detection means S, the transformation means T and the display means A are part of a device 4.

The SCG detection means shown in FIG. 2 and FIG. 3 may, for example, be formed as an acceleration sensor, a pressure sensor or a radar sensor, in particular a Doppler radar sensor, or may comprise such a sensor. The SCG detection means may also be formed as a gyroscope or may comprise such a gyroscope.

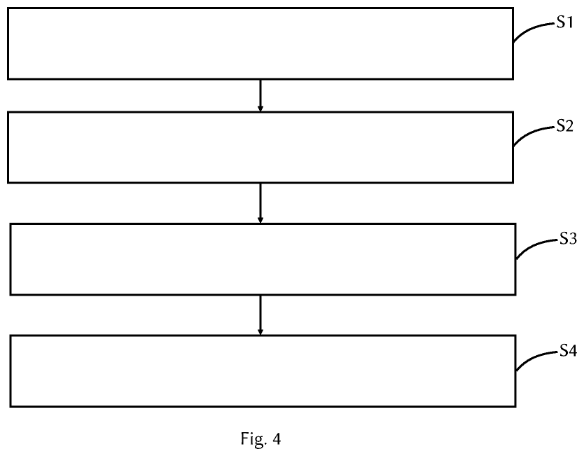

FIG. 4 shows a schematic flow diagram of a method according to the invention. Here, in a detection step S1, an SCG signal 1 is detected, in particular by means of an SCG detection means S, which was explained previously. In an optional filter step S2, the SCG signal 2 detected in this way is filtered, for example high-pass filtered. Also, a so-called trend removal may be carried out in the SCG signal 2. In a transformation step S3, which may be carried out in the transformation means T, the SAG signal is transformed into an ECG signal. Thus, a seismocardiogram may also be transformed into an electrocardiogram. The transformation step S3 may also comprise a plurality of partial transformations. In a post-processing step S4, the ECG signal generated in this way or the electrocardiogram generated in this way is stored, transmitted to at least one further means, and/or visualized, for example on a suitable display means A.

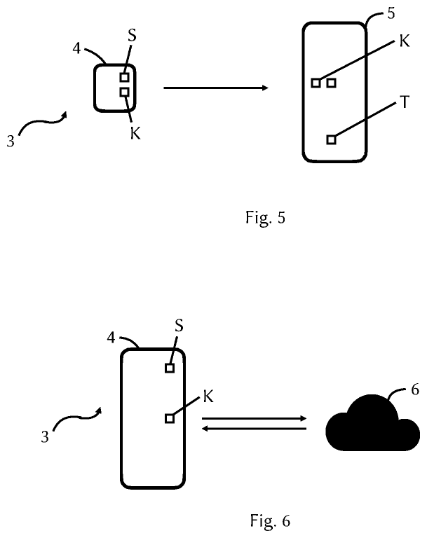

FIG. 5 shows a schematic representation of a system 3 for generating an ECG signal 1 (see FIG. 1) according to a further embodiment. A device 4 is shown, which comprises an SCG detection means S. An SCG signal 2 (see FIG. 1) is detectable by this SCG detection means S. Furthermore, the device comprises a communication means K for data transmission between the device 4 and other devices. This communication means K transmits the generated SCG signal 1 to a HUB device 5. This HUB device 5 has a transformation means T formed as a computing means and a communication means K for receiving the transmitted SCG signals. Furthermore, the transformation of the SCG signal 2 into the ECG signal 1 may be carried out by the HUB device 5. It is then possible that the ECG signal 1 determined in this way is then displayed on a display means (not shown) of the HUB device 5. It may also be stored by a memory means of the HUB device 5, which is not shown, or transmitted further by the communication means K.

FIG. 6 shows a further illustration of a system 3 for generating an ECG signal 1. In contrast to the embodiment shown in FIG. 5, the SCG signals 2 generated by the SCG detection means S are transmitted via the communication means K to a server means 6 which offers so-called cloud-based services. This server means 6 may comprise a transformation means T, not shown, which carries out the transformation of the SCG signals 2 transmitted by the device 4 into ECG signals 1. In FIG. 6 it is shown that the transformed signals, i.e. the ECG signals 1, are transmitted back to the device 4, wherein they may then be received by the communication means K of the device 4. Then, the ECG signal thus obtained may be stored, further processed or visualized by the device 4, for example by a display means A (not shown) of the device 4.

It is possible here that at least one post-processing step is carried out by the HUB means 5 or by the server means 6. In this case, individual, a plurality of but not all, or all of the previously explained post-processing steps may be carried out by the HUB means 5 or the external server means 6.

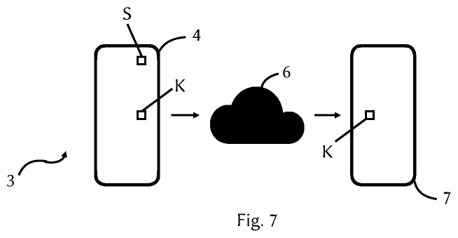

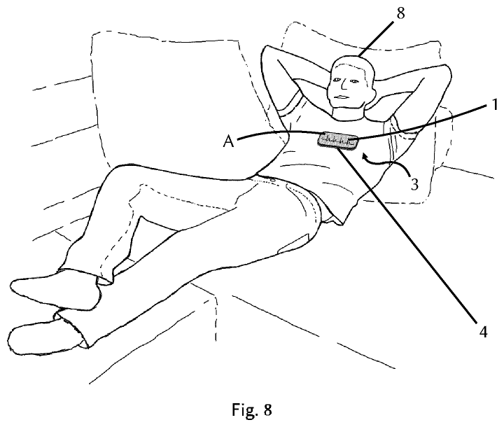

FIG. 7 shows a schematic representation of a system 3 for generating an ECG signal 1 according to a further embodiment of the invention. In contrast to the embodiment shown in FIG. 6, SCG signals 2 detected by the SCG detection means S of the device 4 are transmitted via the communication means K of the device 4 to the server means 6, the transformation means of which then carries out the transformation into ECG signals 1. The ECG signals 1 transformed in this way are then transmitted by the server means 6 to a further device 7, where they are received by means of a communication means K of the further device 7. Furthermore, the ECG signals 1 generated in this way may then be stored in a memory means of the further device 7, processed further by a computing means of the further device 7 or displayed by a display means (not shown) of the further device 7.

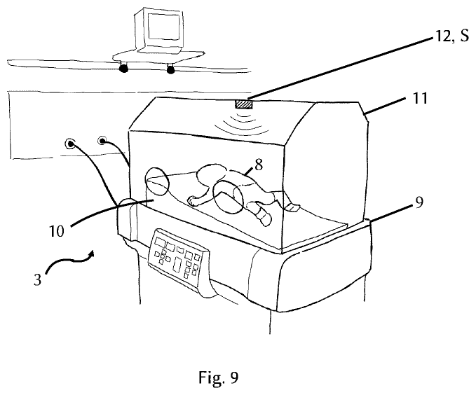

FIG. 8 shows a schematic application of a system 3 (see e.g. FIG. 2) for generating an ECG signal 1. In this case, a device formed as a mobile radio telephone 4, which comprises an SCG detection means S, not shown, and a transformation means T formed as a computing means, is arranged on a chest of a user/patient 8. It is of course conceivable that instead of the mobile radio telephone 4, another device with an SCG detection means S is also used.

By means of the SCG detection means S, SCG signals 2 may then be generated, which are then transformed into ECG signals 1 by the transformation means (not shown) of the device 4 and are then visualized by a display means A of the device 4.

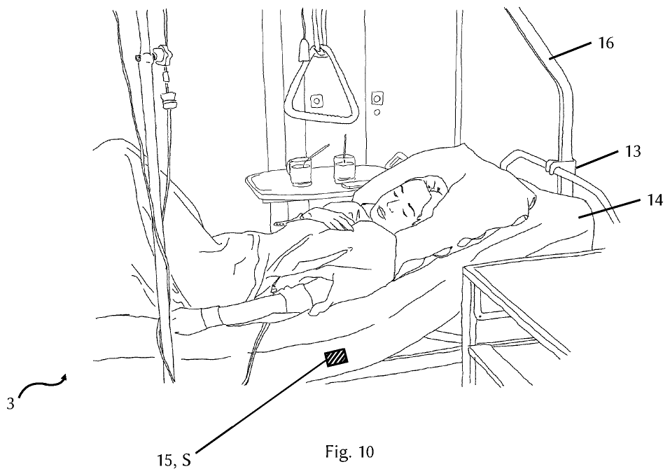

FIG. 9 shows a representation of a system 3 for generating an ECG signal 1 (see FIG. 1) according to a further embodiment. The system 3 comprises an incubator 9, wherein a patient 8, for example a premature baby, lies on a mattress 10 of the incubator 9. Further, the incubator 9 comprises a lid 11 covering the lying space for the patient 8. An SCG detection means S in the form of a Doppler radar sensor 12 is arranged on the lid. This Doppler radar sensor 12 is arranged in such a way that a chest area of the patient 8 lies within the detection range of this sensor 12. Alternatively, it would be possible to arrange, for example, an SCG detection means S formed as a pressure or acceleration sensor in/on the mattress 10 or in/on a floor of the incubator 9 on which the mattress 10 rests. If the patient 8 is a premature baby or a newborn baby, an ECG signal 1 that is completely or highly cleansed of environmental artefacts is able to be generated, in particular by means of suitable filtering methods, since, with the comparatively high heart rate of a newborn baby, a reliable reduction of interfering influences of other persons in the vicinity of the incubator 9 is able to be achieved.

FIG. 10 shows a schematic representation of a system 3 for generating an ECG signal 1 (see FIG. 1) according to a further embodiment. The system 3 comprises a bed 13 with a mattress 14. Furthermore, the system 3 comprises an SCG detection means S formed as a pressure or acceleration sensor 15, which is arranged in/on the mattress 14. Of course, it is also conceivable to use a Doppler radar sensor, wherein this may be arranged on a gallows 16 of the bed 13, for example.

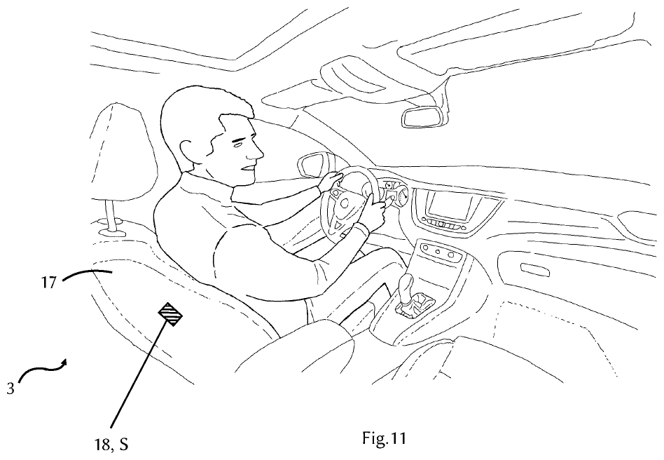

FIG. 11 shows a schematic representation of a system 3 for generating an ECG signal 1 (see FIG. 1) according to a further embodiment. In this case, the system 3 comprises a vehicle seat 17, wherein an SCG detection means S, formed as a pressure or acceleration sensor 18, is arranged in a backrest of the vehicle seat 17. Of course, it is also conceivable to form the SCG detection means S as a Doppler radar sensor and to arrange it in a suitable manner in/on the backrest or at a different location of the vehicle.

The embodiments shown in FIGS. 8, 9, 10, 11 allow, in addition to the normal monitoring of vital data and the normal diagnosis of cardiological pathologies, also a favorable, unbroken as well as electrode-free monitoring and thus also the detection of possibly previously undiagnosed cardiological pathologies, such as intermittent atrial fibrillation.

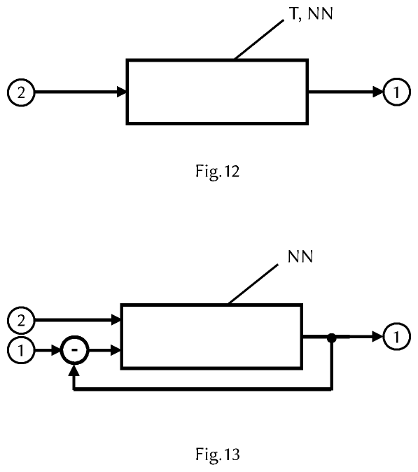

FIG. 12 shows a schematic representation of a method according to the invention in a further embodiment. Here it is shown that SCG signals 2 form input data for a neural network NN, which carries out the transformation of SCG signals into ECG signals 1. Thus, the output signals of the neural network NN are the ECG signals 1 to be generated as proposed. In this case, the transformation means T is formed as a neural network NN, comprises such a network, or may execute functions of a neural network NN.

FIG. 13 shows a schematic representation of the generation/training of the neural network NN shown in FIG. 12. Here, training data in the form of simultaneously detected SCG signals 2 and ECG signals 1 are fed into the neural network NN, wherein parameters of the neural network NN are adapted in such a way that a deviation between the ECG signals 1 generated by the neural network, which are output data of the neural network NN, and ECG signals of the training data set is minimized.

The training dataset may result from a combined measurement of ECG signals, breathing, and seismocardiogram. Such a dataset is available, for example, in the form of a publicly accessible dataset as part of Physiobank. Data from 20 (12 male and 8 female) presumably healthy test subjects were used to test the method. The mean age of the test subjects was 24.4 years (SD±3.10). For the purpose of data acquisition, a Biopac MP36 was used and ECG signals 1 were acquired via the first and second channels and SCG signals 2 via the fourth channel using an accelerometer (LIS344ALH, ST Microelectronics). The test subjects were asked to lie awake and still in the supine position. Three types of recordings were made (basal condition, five minutes; listening to classical music, 50 minutes; control condition, one minute). ECG signals 1 were recorded with a bandwidth between 0.05 Hz and 150 Hz; SCG signals 2 were detected with a bandwidth between 0.5 Hz and 100 Hz. In each channel, sampling was carried out with a sampling rate of 5 kHz.

The following is an example of the architecture of the neural network used and the training data, including their pre-processing, as applied for the testing of the method.

In particular, to run the neural network on embedded devices and smart wearable devices such as phones, a convolutional autoencoder was used to learn the SCG-to-ECG transformation. The autoencoder uses an encoder and a decoder, each with four one-dimensional convolutional layers. In the encoder, the convolutional layers are followed by a ReLU activation function for mapping non-linearity and a max-pooling layer for reducing computing effort, which is used to reduce overfitting and/or to resolve rigid spatial relation. In the encoder, the first convolutional layer starts with 128 filters with a kernel size of 8; with each subsequent layer, the number of filters doubles. The latent space halves the number of filters. In the decoder, the decoder starts with 256 filters in the first convolutional layer. In the second and third layer, the number of filters is halved in each case. The last convolutional layer reduces the number of filters from 64 to 1. Each layer in the decoder consists of an upsampling layer, a convolutional layer, and a ReLU activation function.

SCG and ECG recordings of the dataset were re-sampled at a sampling rate of 100 Hz to match them to the common sampling rates used in acceleration detection, which typically operate between 100 Hz and 200 Hz. This allows for long-term SCG-to-ECG transformation despite the limited computing power of embedded devices. The SCG signal was filtered with a 5-30 Hz fourth-order bandpass Butterworth filter. The signal was then normalized (linear mapping between 0 and 1). Additional filtering of the ECG signals was not performed as they were already pre-filtered.

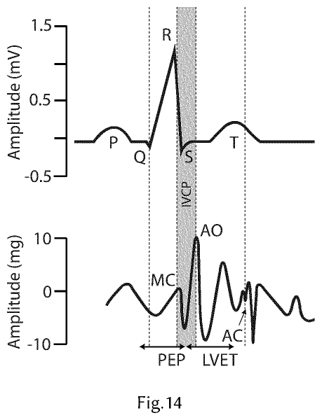

FIG. 14 shows a schematic representation of synchronized ECG and SCG signals, wherein the ECG signal is shown in the top line and the SCG signal is shown in the bottom line.

Prior to training, the weights of the convolutional layers of the model were pre-initialized with a Glorot uniform initialization. The loss function is given by the mean absolute error and is optimized by the Adam optimizer with standard parameters and no regularization term. The label or reference is a ground truth ECG signal (ECGGT), so that the autoencoder learns a mapping from SCG signals 2 to ECGGT signals and then transforms the SCG signal 2 to an ECG signal 1 (ECGT) determined by transformation. In the next step, each 512-value SCG window is fed into the network. The result of the model is a 512-value-long ECGT window, which is adapted to the corresponding ECGGT window via loss optimization.

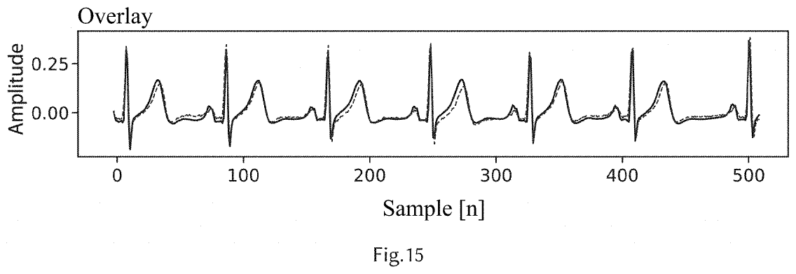

A sliding window technique was used for the training to increase the number of samples and to ensure that the network properly captured the transitions between the windows. Choosing a window size of 512 with an overlap of 87.5% resulted in 4,040 usable windows for each participant. For all 20 participants, the input is therefore reshaped to a tensor 20×512×4040×2. Due to the small number of test subjects, leave-one-out k-fold cross-validation was carried out to assess the generalization performance of the model. Performance was calculated by averaging the 20 folds. To illustrate how the ECGT and ECGGT signals look, FIG. 15 shows the transformation result (ECGT signal), which is represented by a dashed line, of a 400-sample-long segment (user 10 in the dataset) with an overlay of the ECGGT, which is represented by a solid line. The results were evaluated using three different types of metrics: 1) signal-level assessment; 2) feature-level assessment; 3) domain expert assessment.

### Signal-Level Evaluation

Cross-correlations were used to compare the ECGGT with the ECGT. Both signals are highly correlated with a correlation coefficient of r=0.94. To analyze the quality of the signal-level transformation results, a number of appropriate ECG comparison values including the mean square error, normalized mean square error, root mean square error, normalized mean square error and percentage mean square difference were also evaluated. In addition, leave-one-out cross-validation was carried out with all test subjects and the means and standard deviations for each indicator were calculated. Results are shown in Table 1.

### Feature-Level Assessment

For feature-level comparisons, two important ECG features, namely the number of R-peaks and the duration of QRS complexes, were extracted from both signals.

TABLE 1
Mean values (M) and standard deviations (SD) for ECG comparative values

| Parameter | M ± SD |
|---|---|
| Cross-correlation | 0.94 ± 0.05 |
| Mean square error (MSE) | 0.01 ± 0.01 |
| Normalized mean square error (NMSE) | 0.79 ± 0.59 |
| Effective value (RMS) | 0.84 ± 0.30 |
| Normalized root mean square value (NRMS) | 0.09 ± 0.05 |
| Percentage of effective value deviation | 84.4 ± 30.5 |

To identify QRS complexes and R-peaks in the signals, the Pan-Tompkins algorithm was applied. The number of correctly detected R-peaks and the duration of the QRS complexes were used to compare the number of R-peaks and the duration of the QRS complexes. To investigate the differences between the ECGGT and the ECGT, a non-parametric Bland-Altman test was carried out. This Bland-Altman analysis showed, in accordance with the hypothesis, no significant differences between the ECGGT and the ECGT for both the number of R-peaks identified (mean bias=−8.0, 95% CI=−60 to 44, r2=0.97, p=0.56) and the length of QRS complexes (mean bias=−0.34, 95% CI=−1.9 to 1.2, r2=0.02, p=0.12).

### Evaluation by Experts

For this, feedback was collected from 15 cardiologists who examined examples of congruent signals as well as signals with a stronger statistical deviation. The experts were asked to rate the rhythmological and morphological diagnostic value of the signals on a 5-point Likert scale (1—very poor, 2—poor, 3—neutral, 4—good, 5—very good). The average scores for the congruent signals reached 4.87 out of 5 for rhythmological and 4.67 out of 5 for morphological diagnostic value. Even for the signals with lower statistical congruence, the average result was 4.73 out of 5 for the rhythmological and 4.60 out of 5 for the morphological diagnostic value. These results show that the proposed method ensures the determination of an ECG signal 1 with a high reliability and validity. At the signal level, strong correlations between the ECGT and the ECGGT (cross-correlation r=0.94) could be found. At the feature level, comparisons for the number of R-peaks and the length of the QRS complexes demonstrate the agreement of the ECGGT with the ECG signal 1 determined by the transformation applied in accordance with the invention. In general, the data set used provided a high quality of the SCG and ECGGT signals. Nevertheless, some recordings contained motion artefacts that affected both signals. Low-quality ECGGT data and motion artefacts in the SCG data reduce the quality of the ECG signal 1 (ECGT) determined by transformation, influence the number of detected R-peaks in noisy signal sections, and lower the correlation coefficient. On the other hand, artefacts in the ECGGT also reduce the correlation coefficient if the artefact-free SCG signal 2 enabled the determination of a high-quality ECG signal 2. In these cases, the ECG signal 1 as determined according to the invention proves to be better than the recorded ground truth. In particular, in the case of artefacts in the ECGGT signal due to incorrect electrode placement, the ECG signal 1 determined by transformation may provide more accurate results, as it may be generated independently of an electrode connection or correct electrode placement. Systematic feedback from cardiologists also demonstrates the clinical validity and relevance.

In addition, the method proposed according to the invention, which may also be referred to as the Heart.AI method, enables rhythmological pathologies (e.g. atrial fibrillation) to be reliably identified. Furthermore, the contactless applicability of the method, its simple application and the high availability are advantageous. Also advantageous is the possibility to apply the method with SCG detection means in hospital beds or beds in care facilities or even in the home environment. Also advantageous is the ease of use in rural areas where there is often a shortage of general practitioners and in particular specialists. The proposed method may be easily and cost-effectively used in such a scenario for telemedicine applications.

Furthermore, an existing device with a means capable of detecting an SCG signal 2, e.g. an acceleration sensor or a gyroscope, may be made able to carry out the proposed method by way of a software update. Thus, the functionality provided by the method may be retrofitted on a wide range of devices, which results in a broad applicability of the method. A further advantage is that a simple and reliable permanent detection of precordial movements (SCG signal) is possible, which then also enables the permanent and reliable determination of an ECG signal, in particular in a period longer than 24 hours. It is also advantageous that the required sensor technology is inexpensive and that the required sensors are already installed in many usable devices and may therefore—as explained above—be used for carrying out the method. The proposed method may also be used to subsequently transform already generated SCG signals 2 into ECG signals 1. This is particularly beneficial for scientific investigations.

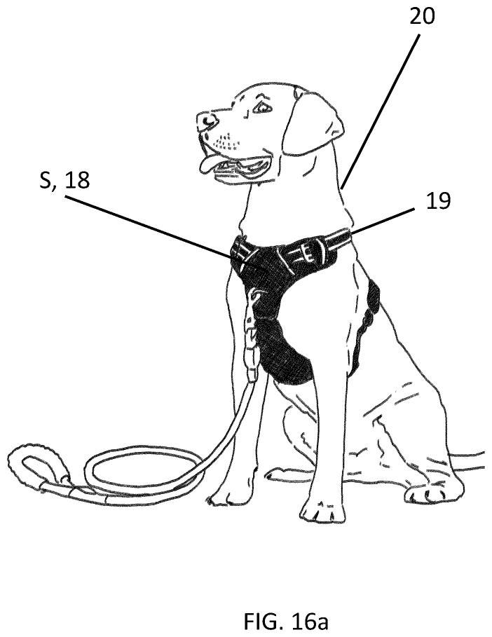

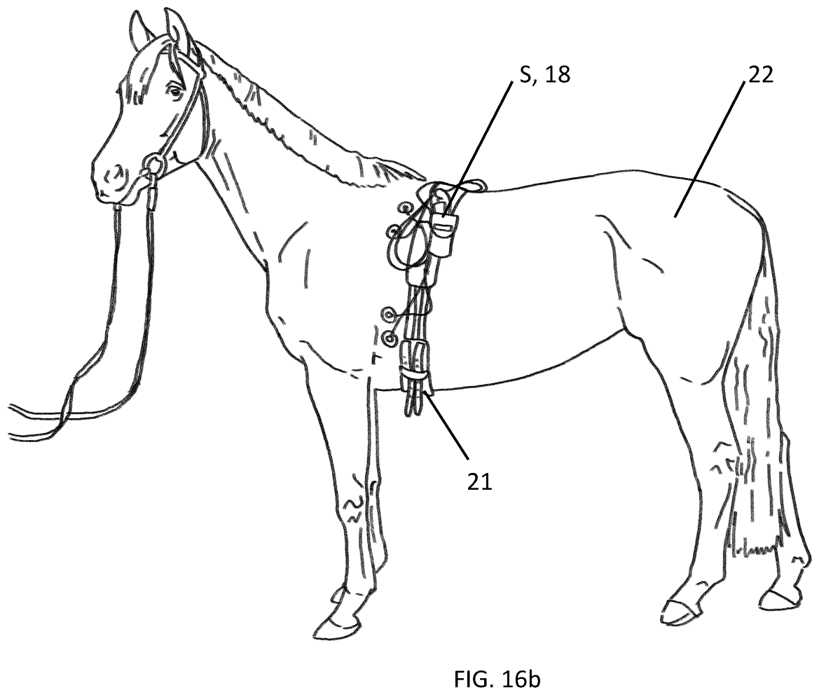

FIG. 16a shows a schematic representation of a dog harness 19 with an SCG detection means S of a system 3 for generating an ECG signal 1 (see FIG. 1), wherein the SCG detection means S is formed as an acceleration sensor 18. It is shown that the SCG detection means S is arranged in a region of the dog harness 19 which rests against a chest region of the dog 20 wearing the dog harness 19 in the intended manner.

FIG. 16b shows a schematic representation of a horse halter 21 with an SCG detection means S of a system 3 for generating an ECG signal 1 (see FIG. 1), wherein the SCG detection means S is formed as an acceleration sensor 18. It is shown that the SCG detection means S is arranged in a region of the halter 21 which rests against an upper back region of the horse 22 wearing the halter 21 in the intended manner. However, it is also conceivable that the SCG detection means S is arranged in an area of the halter 21 that rests against the belly or chest area of the horse 22 wearing the halter 21 in the intended manner.

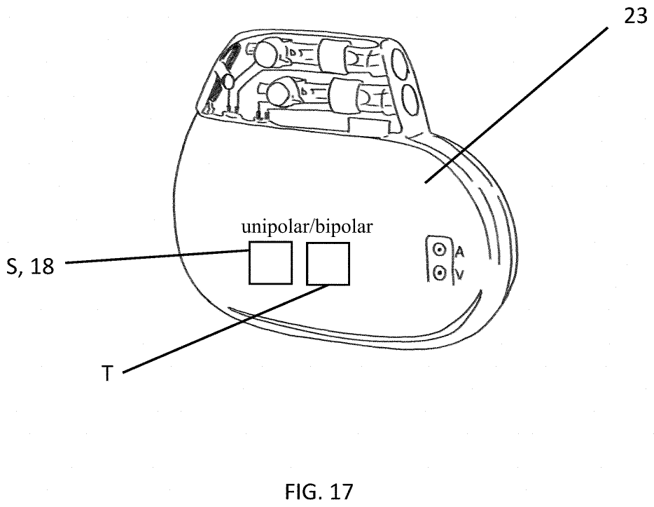

FIG. 17 shows a schematic representation of a pacemaker 23 with a system 3 for generating an ECG signal 1. A rate-adaptive pacemaker 23 is shown, which comprises an SCG detection means S, which is formed as an acceleration sensor 18. Further, the pacemaker 22 comprises a transformation means T. Not shown is a communication means K of the pacemaker 23, which is able to transmit the ECG signal 1 determined by transformation to a means external to the body, for example a display means A or a server means 6. However, it is not mandatory that the pacemaker 23 comprises the transformation means T. Thus, it is also possible that the pacemaker 23 does not comprise a transformation means T and the output signals (raw signals) of the SCG detection means S are transmitted to a computing means external to the pacemaker, e.g. via the communication means K.

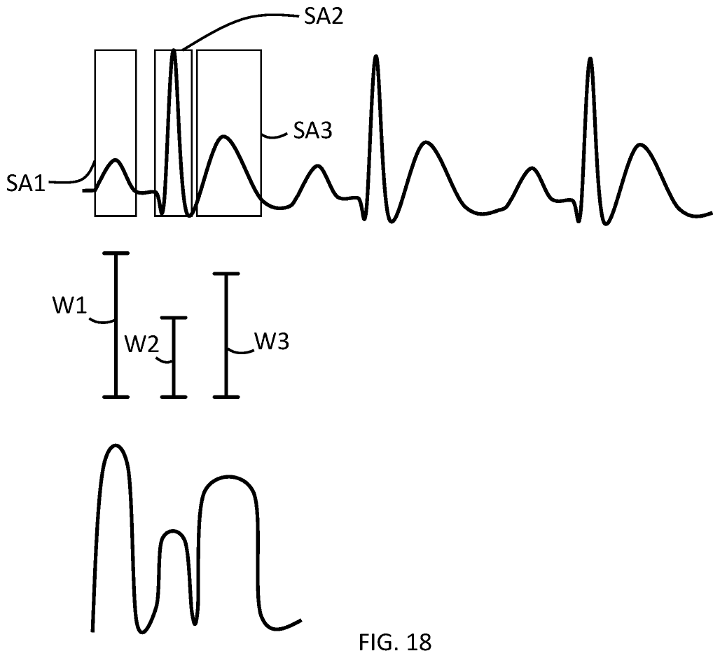

FIG. 18 shows an example representation of weightings of different signal sections for the evaluation of an error function. The top line shows an ECG signal. Three different signal sections SA1, SA2, SA3 are shown in the ECG signal, wherein the different signal sections are enclosed by a rectangle. The first signal section SA1 is a P-wave signal section, the second signal section SA2 is a QRS complex signal section, and the third signal section SA3 is a T-wave signal section. The second, middle row shows weighting factors w1, w2, w3 assigned to the individual signal sections SA1, SA2, SA3. Thus, a first weighting factor w1 is assigned to the first signal section SA1, a second weighting factor w2 to the second signal section SA2, and a third weighting factor w3 to the third signal section SA3. It can be seen that the first weighting factor w1 is greater than the second and the third weighting factor w2, w3, the third weighting factor w3 being greater than the second weighting factor w2. It is possible that the weighting factors are greater than one. However, it is also possible that all weighting factors w1, w2, w3 are equal and greater than one, whereby the signal sections SA1, SA2, SA3 that are relevant for an ECG are weighted higher in relation to the remaining, non-relevant signal sections. The third, lower line shows a signal curve of the weighted ECG signal, wherein the amplitude of the ECG signal in the first signal section SA1 has been weighted, in particular multiplied, by the first weighting factor w1, in the second signal section SA2 by the second weighting factor w2, and in the third signal section SA3 by the third weighting factor w3.

The weighting may also be carried out by convolution of the ECG signal with a window function. This weighting may be used in particular to carry out amplitude compensation. In this way, it is possible to avoid large signal changes being weighted higher than smaller changes, which is the case, for example, when determining the deviation with the mean square error method. In the case of the ECG signal, however, small elevations (e.g. the P-wave enclosed in the first signal section SA1) contain important information. It is conceivable that in this way different signal sections of an ECG signal 1 determined by transformation as well as different signal sections of a reference ECG signal are weighted, and after the weighting the deviation between the weighted signals is then determined in order to train the model for the transformation, in particular a neural network. The phrase “at least one of A, B, and C” should be construed to mean a logical (A OR B OR C), using a non-exclusive logical OR, and should not be construed to mean “at least one of A, at least one of B, and at least one of C.”

1-20. (canceled)

21. A method for generating electrocardiogram (ECG) signals comprising:
detecting at least one cardiac motion induced signal, wherein the at least one cardiac motion induced signal is a seismocardiography (SCG) signal; and
transforming the at least one detected cardiac motion induced signal into at least one ECG signal, wherein a plurality of channel-specific signals of a multi-channel ECG signal are determined by the transformation from the at least one SCG signal.

22. The method of claim 21 wherein all the plurality of channel-specific signals of the multi-channel ECG signal are determined by the transformation from the at least one SCG signal.

23. The method of claim 21 wherein the transformation is performed using a model generated by machine learning.

24. The method of claim 23 wherein the transformation is performed using a neural network.

25. The method of claim 23 wherein the transformation is performed using at least one of an autoencoder, a convolutional neural network, a long short-term memory (LSTM) network, and a neural transformer network.

26. The method of claim 21 wherein the transformation is carried out by at least one of a predetermined mathematical model and a predetermined transformation function.

27. The method of claim 23 wherein:
generating the model includes evaluating an error function for determining a deviation between the at least one ECG signal and a reference ECG signal; and
during the evaluation of the error function, different weights are applied to different signal sections of at least one of the reference ECG signal, the deviation, and the at least one ECG signal.

28. The method of claim 21 wherein the at least one cardiac motion induced signal is detected contactlessly.

29. The method of claim 21 further comprising filtering the at least one cardiac motion induced signal prior to the transformation, such that the filtered cardiac motion induced signal is transformed into the at least one ECG signal.

30. The method of claim 21 wherein:
the at least one cardiac motion induced signal is generated by a detector of a device; and
the transformation is carried out by a processor of the device.

31. The method of claim 21 wherein:
the at least one cardiac motion induced signal is generated by a detector of a device; and
the cardiac motion induced signal is transmitted to a processor of a further device and the transformation is carried out by the processor of the further device.

32. The method of claim 21 wherein:
the at least one cardiac motion induced signal is generated by a detector of a device; and
the at least one ECG signal is displayed on a display of the device.

33. The method of claim 21 wherein:
the at least one cardiac motion induced signal is generated by a detector of a device; and
the at least one cardiac motion induced signal is transmitted to a display of a further device and is displayed by the display of the further device.

34. The method of claim 21 further comprising:
prior to the transformation of the at least one cardiac motion induced signal, performing a functional test of a detector,
wherein the cardiac motion induced signal is transformed only if a specified functional capability is detected.

35. The method of claim 21 further comprising:
prior to the transformation of the at least one cardiac motion induced signal, determining a signal quality of the detected signal,
wherein the cardiac motion induced signal is transformed only if the signal quality is greater than or equal to a specified measure.

36. The method of claim 21 further comprising:
prior to the transformation of the at least one cardiac motion induced signal, determining an arrangement of a detector relative to a heart,
wherein the cardiac motion induced signal is transformed only if the arrangement corresponds to a specified arrangement or deviates therefrom by less than a specified measure.

37. A system for generating electrocardiogram (ECG) signals comprising:
a detector configured to detect at least one cardiac motion induced signal, wherein the at least one cardiac motion induced signal is a seismocardiography (SCG) signal; and
a processor configured to transform the at least one detected cardiac motion induced signal into at least one ECG signal, wherein a plurality of channel-specific signals of a multi-channel ECG signal are determined by the transformation from the at least one SCG signal.

38. The system of claim 37 wherein the detector is integrated into at least one of an incubator, a bed, a vehicle seat, a pacemaker, and an animal accessory.

39. The system of claim 37 wherein all the plurality of channel-specific signals of the multi-channel ECG signal are determined by the transformation from the at least one SCG signal.

40. The system of claim 37 wherein:
the transformation is performed using a model generated by machine learning; and
the model is based on at least one of an autoencoder, a convolutional neural network, a long short-term memory (LSTM) network, and a neural transformer network.
